(19) United States
(12) Patent Application Publication
(10) Pub. No.: US 2013/0060111 A1
(43) Pub. Date: Mar. 7, 2013

(54) DUAL PAD FOR HYGIENE AND DETECTING CONDITIONS

(71) Applicants: Alejandro Vargas, (US); Juan A. Perez, (US); Miguel A. Garrido, (US)

(72) Inventors: Alejandro Vargas, (US); Juan A. Perez, (US); Miguel A. Garrido, (US)

(21) Appl. No.: 13/226,932

(22) Filed: Sep. 7, 2011

(51) Int. Cl. A61B 5/00

(52) U.S. Cl. 600/365; 600/309

(57) ABSTRACT

The present invention provides a hygienic pad enabled not only to collect human discharges when a man or a woman uses the pad, but also to detect reactants contained in the discharges. The detected reactants are indicators of medical conditions. Means to detect the reactants are incorporated into the hygienic pad.

### BACKGROUND OF THE INVENTION

1. Field of the Invention

The present invention is related to a hygienic pad that has been enabled to detect reactants which are indicators of medical conditions.

2. Description of Prior Art

Detecting medical conditions, e.g. pregnancy, diabetes etc., usually requires specialized kits. However there are no means to detect such medical conditions when everyday hygienic products are used.

The present invention contributes to overcome the mentioned limitations of the prior art.

### SUMMARY OF THE INVENTION

The present invention provides a hygienic pad enabled not only to collect human discharges when a man or a woman uses the pad, but also to detect reactants contained in the discharges. The detected reactants are indicators of medical conditions. Means to detect the reactants are incorporated into the hygienic pad.

Specifically, the present invention provides an hygienic pad for diagnostic of a medical condition, wherein the pad comprises:
A. A hygienic pad; and,
B. Means to detect a reactant, where the means to detect the reactant are incorporated into the hygienic pad, wherein the reactant is contained in human discharges released into the hygienic pad, wherein said discharges are in the hygienic pad because of use of the hygienic pad by a human being (a man or a woman), wherein the reactant at a certain concentration is indicative of the medical condition.

In a preferred version of the hygienic pad of the present invention, the means to detect the reactant is at least one diagnostics strip inserted into the hygienic pad.

In another version of the hygienic pad of the present invention, the means to detect the reactant are diagnostic microspheres inserted into the hygienic pad.

In an aspect of the hygienic pad of the present invention, the reactant to be detected is human chorionic gonadotropin.

In another embodiment of the hygienic pad of the present invention, the reactant to be detected is glucose.

In other embodiment of the hygienic pad of the present invention, the reactant to be detected is a human hormone.

In one more embodiment of the hygienic pad of the present invention, the reactant to be detected is an antibody against a germ that infects humans.

In an additional embodiment of the hygienic pad of the present invention, the reactant to be detected is a nucleic acid of a germ that infects humans.

In a further embodiment of the hygienic pad of the present invention, the reactant to be detected is a protein of a germ that infects humans.

Additional objectives and advantages of the present invention will be more evident in the detailed description of the invention and the claims.

### BRIEF DESCRIPTION OF THE DRAWINGS

FIG. 1. shows an illustration of an embodiment of the hygienic pad of the present invention wherein there are seven diagnostic strips incorporated into the hygienic pad.

FIG. 2. shows an illustration of an embodiment of the hygienic pad of the present invention wherein there are six diagnostic strips incorporated into the hygienic pad.

### DETAILED DESCRIPTION OF THE INVENTION

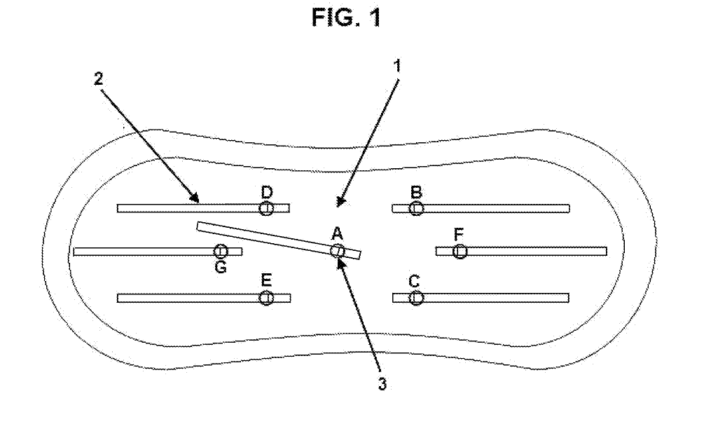

FIG. 1 shows an illustration the preferred embodiment of an hygienic pad for diagnostic of a medical condition, wherein the pad comprises:
A. A hygienic pad (1); and,
B. Means, which in a preferred case are diagnostic strips (2, 3), to detect a reactant, where the diagnostic strips (2, 3) to detect the reactant are incorporated into the hygienic pad (1), wherein the reactant is contained in human discharges released into the hygienic pad, wherein said discharges are in the hygienic pad because of use of the hygienic pad by a man or a woman, wherein the reactant at a certain concentration is indicative of the medical condition.

For purposes of the present invention, the term “hygienic pad” refers to any kind of hygienic or sanitary pad to be used with or without underwear by a man or woman to cover his/her genital and/or anal area, wherein the hygienic or sanitary pad covers the genital and/or anal area, wherein the hygienic pad absorbs the man or woman discharges; and,

wherein the hygienic pad is very clean or preferably sterile.

For purposes of the present invention, the term “discharge” includes any, or combination of any, of the following:
Urine.
Genital secretions or excretions.
Wounds discharges (blood, pus, etc.).
Blood.
Menstrual discharge.
Any kind of gland secretion.
Any kind of mucosal secretion.
Anal excretions.
Vaginal discharges.
Semen.
Any kind of liquid, semi-liquid, gel-like discharge, cell-material produced to the exterior of the genital area and/or anal area by a human tissue and/or organ, wherein said discharge can be absorbed by a hygienic pad as defined in paragraph 0016.

In a preferred version of the hygienic pad (1) of the present invention, the means to detect the reactant is at least one diagnostics strip (2, 3) inserted or incorporated into the hygienic pad. In the preferred example the diagnostic strip has a marker that becomes visible when a reactant contained in a human discharge contacts certain part (3) of the diagnostic strip. Examples of diagnostic strips include the commercially available strips used to detect human chorionic gonadotropin in urine for pregnancy testing; commercially diagnostic strips to measure glucose concentration in blood or urine, etc.

In another version of the hygienic pad of the present invention, the means to detect the reactant are diagnostic microspheres inserted into the hygienic pad.

For purposes of the present invention, “means to detect a reactant” contained in a human discharge comprises a molecular marker; and any, or combination of any, of the following:
Diagnostics strips to which an antibody, and/or a molecular protein, and/or a nucleic acid fragment, and/or a lipid, and/or an organic or inorganic chemical molecule can be attached, etc.
Microspheres to which an antibody, and/or a molecular protein, and/or a nucleic acid fragment, and/or a lipid, and/or an organic or inorganic chemical molecule can be attached, etc.
Fibers (cellulose, etc.) to which antibody, and/or a molecular protein, and/or a nucleic acid fragment, and/or a lipid, and/or an organic or inorganic chemical molecule can be attached, etc.
Any kind of physical material, to which antibody, and/or a molecular protein, and/or a nucleic acid fragment, and/or a lipid, and/or an organic or inorganic chemical molecule can be attached, etc.
   Wherein the antibody, and/or a molecular protein, and/or a nucleic acid fragment, and/or a lipid, and/or an organic or inorganic chemical molecule interacts with the reactant contained in the human discharge, forming an antibody, and/or a molecular protein, and/or a nucleic acid fragment, and/or a lipid, and/or an organic or inorganic chemical molecule complex with the reactant, and wherein the reaction of the complex with the marker results in a visible color to the naked eye, wherein the marker preferably attaches to the complex.

In a preferred embodiment of the invention “the means to detect the reactant” is comprised by a first antibody attached to the diagnostic strip (the first antibody is specific for the reactant); and a second antibody also attached to the diagnostic strip which is specific against the first antibody, wherein the second antibody will be (for the purposes of the present invention) the marker since the reaction of the second antibody with the first antibody-reactant complex produces visible color. This “means to detect the reactant” mechanism is known as Enzyme-linked immunosorbent assay (ELISA), used in commercially available pregnancy test diagnostic strips.

For purposes of the present invention, the term “reactant” includes any, or combination of any, of the following:
Antibodies against any microorganism or germ (virus, bacteria, protozoan, etc.) that infects the human body, wherein the antibody is produced by the human body.
Nucleic acid of a microorganism or a germ that infects the human body.
Proteins produced by a microorganisms or a germ that infect the human body.
Glucose.
A human hormone.
Any substance or material produced by the human body as a result of a medical condition.

For purposes of the present invention, the term “medical condition” includes any, or a combination of any, of the following:
A microorganism or germ caused infection and/or disease to the human body.
Pregnancy.
A status wherein substances (metabolites, hormones, chemicals, etc.) in a living human body are within abnormal parameters.
A disease of any origin.
Any condition of a living human body which results in a substance or material present in a human body discharge, wherein the concentration of said substance or material in the discharge is not normal (most frequently above normal concentration parameters), and wherein the substance or material constitutes the reactant as defined in paragraph 0021.

Although the description presents preferred embodiments of the present invention, additional changes may be made in the form and disposition of the parts without deviating from the ideas and basic principles encompassed by the claims.

### EXAMPLES

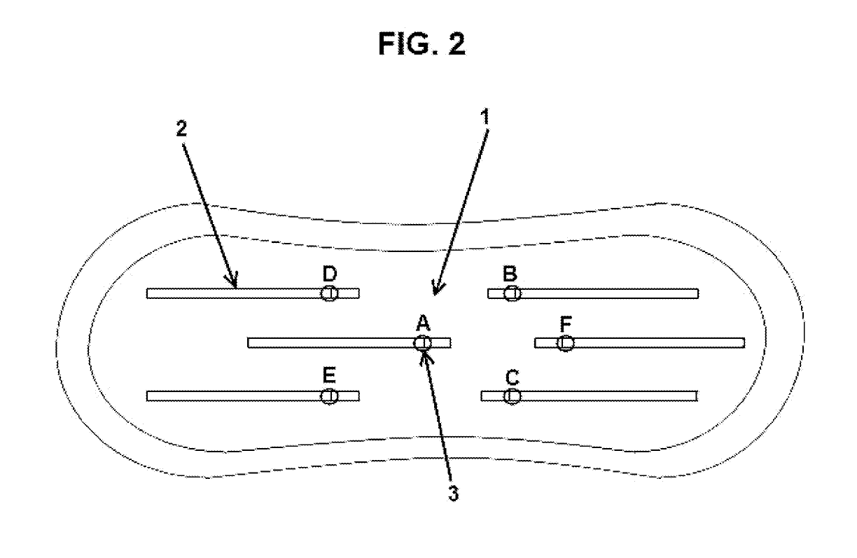

As it is shown if FIG. 1, commercially pregnancy test diagnostic strips were inserted into commercially available feminine hygienic pads. Five of the hygienic pads with the inserted pregnancy test strips were given to a woman who was in the second trimester of pregnancy. The pregnant woman used non-consecutively each one of the five hygienic pads with pregnancy test strips for a period of 12 hours. The five of the hygienic pads with the pregnancy test diagnostic strips, used by the woman, were examined for positive results for pregnancy. A positive result was determined to be appearance of two colored bands in one region (the area of the circle shown by 3 in FIGS. 1 and 2) of the pregnancy test diagnostic strip.

The following table shows the results in the five examined hygienic pads with pregnancy test diagnostic strips:

TABLE 1
Results of the pregnancy test according to the position of
the pregnancy test diagnostic strip on the hygienic pad

| | PAD | PAD | PAD | PAD | PAD |
|---|---|---|---|---|---|
| | No. 1 | No. 2 | No. 3 | No. 4 | No. 5 |
| | 19 May | 19 May | 23 May | 23 May | 24 May |
| POSI- | 2011 | 2011 | 2011 | 2011 | 2011 |
| TION* | day | night | day | night | night |
| A | | + | | | |
| B | | | + | | |
| C | | | + | | |
| D | + | | + | | |
| E | | | + | + | + |
| F | + | + | | | |
| G | | + | | | |

*The positions A, B, C, D, E, F, and G are illustrated in FIG. 1.

These results indicate that the hygienic pads with the pregnancy test diagnostic strips successfully detected, in all cases, positively the reactant, which indicates pregnancy. At least of the pregnancy test diagnostic strips inserted in the hygienic pad detected the reactant in accord with the medical condition, pregnancy, of the woman.

According to the results, hygienic pads with incorporated means (in this case diagnostic strips) to detect a reactant that indicates a medical condition can be used to screen for the medical condition, e.g., pregnancy, glucose levels indicating diabetes, presence of antibodies against a germ causing infection, proteins and/or nucleic acids derived from a germ causing an infection (sexually transmitted disease germs), etc.

1. An hygienic pad for diagnostic of a medical condition, wherein the pad comprises:
A. A hygienic pad; and,
B. Means to detect a reactant, where the means to detect the reactant are incorporated into the hygienic pad, wherein the reactant is contained in human discharges released into the hygienic pad, wherein said discharges are in the hygienic pad because of use of the hygienic pad by a human being, wherein the reactant at a certain concentration is indicative of the medical condition.

2. The Hygienic pad of claim 1, wherein the means to detect the reactant is at least one diagnostics strip inserted into the hygienic pad.

3. The Hygienic pad of claim 1, wherein the means to detect the reactant are diagnostic microspheres inserted into the hygienic pad.

4. The hygienic pad of claim 1, wherein the reactant to be detected is human chorionic gonadotropin.

5. The hygienic pad of claim 1, wherein the reactant to be detected is glucose.

6. The hygienic pad of claim 1, wherein the reactant to be detected is a human hormone.

7. The hygienic pad of claim 1, wherein the reactant to be detected is an antibody against a germ that infects humans.

8. The hygienic pad of claim 1, wherein the reactant to be detected is a nucleic acid of a germ that infects humans.

9. The hygienic pad of claim 1, wherein the reactant to be detected is a protein of a germ that infects humans.
